SEQUENCE LISTING (33 sequences)

SEQ ID NO: 1 - protein, 15 aa, Artificial Sequence
  CDRL1 of hPD-1.09A
RASKGVSTSGYSYLH

SEQ ID NO: 2 - protein, 7 aa, Artificial Sequence
  CDRL2 of hPD-1.09A
LASYLES

SEQ ID NO: 3 - protein, 9 aa, Artificial Sequence
  CDRL3 of hPD-1.09A
QHSRDLPLT

SEQ ID NO: 4 - protein, 111 aa, Artificial Sequence [sequence omitted]

SEQ ID NO: 5 - protein, 218 aa, Artificial Sequence
  Light chain of pembrolizumab
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, Artificial Sequence
  CDRH1 of hPD-1.09A
NYYMY

SEQ ID NO: 7 - protein, 17 aa, Artificial Sequence
  CDRH2 of hPD-1.09A
GINPSNGGTNFNEKFKN

SEQ ID NO: 8 - protein, 11 aa, Artificial Sequence
  CDRH3 of hPD-1.09A
RDYRFDMGFDY

SEQ ID NO: 9 - protein, 120 aa, Artificial Sequence [sequence omitted]

SEQ ID NO: 10 - protein, 447 aa, Artificial Sequence
  Heavy chain of pembrolizumab
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 9 aa, Artificial Sequence
  VH CDR1
GTFSSEGIS

SEQ ID NO: 12 - protein, 17 aa, Artificial Sequence
  VH CDR2
SILPIFGTANYAQKFQG

SEQ ID NO: 13 - protein, 14 aa, Artificial Sequence
  VH CDR3
AREAGYYRYRYFDL

SEQ ID NO: 14 - protein, 11 aa, Artificial Sequence
  VL CDR1
RASQSVSSNLA

SEQ ID NO: 15 - protein, 7 aa, Artificial Sequence
  VL CDR2
GASTRAT

SEQ ID NO: 16 - protein, 9 aa, Artificial Sequence
  VL CDR3
QQHALWPLT

SEQ ID NO: 17 - protein, 26 aa, Artificial Sequence
  VH FR1
QVQLVQSGAEVKKPGSSVKVSCKASG

SEQ ID NO: 18 - protein, 14 aa, Artificial Sequence
  VH FR2
WVRQAPGQGLEWMG

SEQ ID NO: 19 - protein, 30 aa, Artificial Sequence
  VH FR3
RVTITADESTSTAYMELSSLRSEDTAVYYC

SEQ ID NO: 20 - protein, 11 aa, Artificial Sequence
  VH FR4
WGRGTLVTVSS

SEQ ID NO: 21 - DNA, 364 bp, Artificial Sequence
  VH DNA
caagtgcagttggtgcagtccggagccgaagtcaagaagcccgggtcgagcgtgaaagtgtcctgcaaggcttctggtggaaccttctcaagcgaagggatcagctgggtcagacaggcgccgggccagggtctggagtggatgggttccattctcccgatcttcggaaccgccaattacgcccagaagttccagggtcgcgtgaccatcaccgccgacgaaagcacctcgacggcctatatggaattgtcgtccctgcggtcggaagatacagcggtgtactactgtgcgcgggaagccgggtactaccgctaccgctacttcgatctgtggggaaggggaactctcgtgactgtgtcgagcg

SEQ ID NO: 22 - protein, 121 aa, Artificial Sequence
  VH Protein
QVQLVQSGAEVKKPGSSVKVSCKASGGTFSSEGISWVRQAPGQGLEWMGSILPIFGTANYAQKFQGRVTITADESTSTAYMELSSLRSEDTAVYYCAREAGYYRYRYFDLWGRGTLVTVSS

SEQ ID NO: 23 - protein, 23 aa, Artificial Sequence
  VL FR1
EIVMTQSPATLSVSPGERATLSC

SEQ ID NO: 24 - protein, 15 aa, Artificial Sequence
  VL FR2
WYQQKPGQAPRLLIY

SEQ ID NO: 25 - protein, 32 aa, Artificial Sequence
  VL FR3
GIPARFSGSGSGTEFTLTISSLQSEDFAVYYC

SEQ ID NO: 26 - protein, 10 aa, Artificial Sequence
  VL FR4
FGGGTKVEIK

SEQ ID NO: 27 - DNA, 321 bp, Artificial Sequence
  VL DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagcacgccctctggcctctcacttttggcggagggaccaaggttgagatcaaa

SEQ ID NO: 28 - protein, 107 aa, Artificial Sequence
  VL Protein
EIVMTQSPATLSVSPGERATLSCRASQSVSSNLAWYQQKPGQAPRLLIYGASTRATGIPARFSGSGSGTEFTLTISSLQSEDFAVYYCQQHALWPLTFGGGTKVEIK

SEQ ID NO: 29 - protein, 447 aa, Artificial Sequence
  Heavy Chain Protein
QVQLVQSGAEVKKPGSSVKVSCKASGGTFSSEGISWVRQAPGQGLEWMGSILPIFGTANYAQKFQGRVTITADESTSTAYMELSSLRSEDTAVYYCAREAGYYRYRYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - DNA, 1341 bp, Artificial Sequence
  Heavy Chain DNA
caagtgcagttggtgcagtccggagccgaagtcaagaagcccgggtcgagcgtgaaagtgtcctgcaaggcttctggtggaaccttctcaagcgaagggatcagctgggtcagacaggcgccgggccagggtctggagtggatgggttccattctcccgatcttcggaaccgccaattacgcccagaagttccagggtcgcgtgaccatcaccgccgacgaaagcacctcgacggcctatatggaattgtcgtccctgcggtcggaagatacagcggtgtactactgtgcgcgggaagccgggtactaccgctaccgctacttcgatctgtggggaaggggaactctcgtgactgtgtcgagcgccagcaccaagggacccagcgtgttcccgctggccccttgttcacgatccacttccgaaagcaccgctgcccttggctgccttgtcaaggactacttccctgagcccgtcactgtgtcgtggaacagcggagctctgacctccggcgtccacaccttcccggctgtgctccagtcctccggcctgtactcactgtcctcggtggtcaccgtgccctcctcctccctcggtaccaagacttatacctgcaacgtggaccacaagccctccaacaccaaagtggataagagagtggagagcaaatacggacctccctgccctccttgccctgcgcctgagtttctgggcggaccatccgtctttctgttcccaccgaagcccaaggacaccctcatgatctcccggacccccgaagtgacctgtgtggtggtggacgtgtcacaggaggaccctgaagtgcagtttaattggtacgtcgacggcgtggaagtgcataacgcaaagaccaagccgcgggaggaacagttcaactcaacctaccgcgtggtgtccgtgctgactgtgctgcaccaggactggctgaacggaaaggagtataagtgcaaagtctccaacaagggactgccgagcagcatcgagaaaaccatttcaaaagccaagggccagccgagagagccccaagtgtacactctgccgccgagccaagaggaaatgaccaagaaccaagtgtccctcacttgcctggtcaagggcttctacccgtcggacatcgccgtggagtgggaaagcaacggccagccggaaaacaactacaagactacccctcccgtcctcgactccgacgggtccttcttcctctactcccggctgactgtggataagtcacggtggcaggagggaaacgtgttctcgtgctccgtgatgcacgaagccctgcacaaccactacacgcagaagtccctgtccttgtccctgggg

SEQ ID NO: 31 - protein, 214 aa, Artificial Sequence
  Light Chain Protein
EIVMTQSPATLSVSPGERATLSCRASQSVSSNLAWYQQKPGQAPRLLIYGASTRATGIPARFSGSGSGTEFTLTISSLQSEDFAVYYCQQHALWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - DNA, 642 bp, Artificial Sequence
  Light Chain DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagcacgccctctggcctctcacttttggcggagggaccaaggttgagatcaaacgtacggtggccgctccctccgtgttcatcttcccaccctccgacgagcagctgaagtccggcaccgcctccgtcgtgtgcctgctgaacaacttctaccctcgcgaggccaaagtgcagtggaaagtggacaacgccctgcagtccggcaactcccaggaatccgtcaccgagcaggactccaaggacagcacctactccctgtcctccaccctgaccctgtccaaggccgactacgagaagcacaaagtgtacgcctgcgaagtgacccaccagggcctgtccagccccgtgaccaagtccttcaaccggggcgagtgc

SEQ ID NO: 33 - DNA, 1431 bp, Artificial Sequence
  Heavy Chain DNA (5′ and 3′ EcoRV restriction sites Kozaksequence; signal sequence
gatatcgccaccatggcctctccagctcagctgctgtttctgctgctgctgtggctgcctgacggcgtgcacgcacaagtgcagttggtgcagtccggagccgaagtcaagaagcccgggtcgagcgtgaaagtgtcctgcaaggcttctggtggaaccttctcaagcgaagggatcagctgggtcagacaggcgccgggccagggtctggagtggatgggttccattctcccgatcttcggaaccgccaattacgcccagaagttccagggtcgcgtgaccatcaccgccgacgaaagcacctcgacggcctatatggaattgtcgtccctgcggtcggaagatacagcggtgtactactgtgcgcgggaagccgggtactaccgctaccgctacttcgatctgtggggaaggggaactctcgtgactgtgtcgagcgccagcaccaagggacccagcgtgttcccgctggccccttgttcacgatccacttccgaaagcaccgctgcccttggctgccttgtcaaggactacttccctgagcccgtcactgtgtcgtggaacagcggagctctgacctccggcgtccacaccttcccggctgtgctccagtcctccggcctgtactcactgtcctcggtggtcaccgtgccctcctcctccctcggtaccaagacttatacctgcaacgtggaccacaagccctccaacaccaaagtggataagagagtggagagcaaatacggacctccctgccctccttgccctgcgcctgagtttctgggcggaccatccgtctttctgttcccaccgaagcccaaggacaccctcatgatctcccggacccccgaagtgacctgtgtggtggtggacgtgtcacaggaggaccctgaagtgcagtttaattggtacgtcgacggcgtggaagtgcataacgcaaagaccaagccgcgggaggaacagttcaactcaacctaccgcgtggtgtccgtgctgactgtgctgcaccaggactggctgaacggaaaggagtataagtgcaaagtctccaacaagggactgccgagcagcatcgagaaaaccatttcaaaagccaagggccagccgagagagccccaagtgtacactctgccgccgagccaagaggaaatgaccaagaaccaagtgtccctcacttgcctggtcaagggcttctacccgtcggacatcgccgtggagtgggaaagcaacggccagccggaaaacaactacaagactacccctcccgtcctcgactccgacgggtccttcttcctctactcccggctgactgtggataagtcacggtggcaggagggaaacgtgttctcgtgctccgtgatgcacgaagccctgcacaaccactacacgcagaagtccctgtccttgtccctggggaagtaatgagatatc

(19) United States
(12) Patent Application Publication
(10) Pub. No.: US 2023/0036592 A1
(43) Pub. Date: Feb. 2, 2023

(54) METHODS OF TREATING CANCER WITH AN ANTI-CD39 ANTIBODY AND PEMBROLIZUMAB

(71) Applicants: Surface Oncology, Inc., Cambridge, MA (US); MSD International GmbH, Luzern (CH)

(72) Inventors: Alison O'NEILL, Cambridge, MA (US); Jou-Ku CHUNG, Cambridge, MA (US); Robert ROSS, Cambridge, MA (US); Andrew LAKE, Cambridge, MA (US)

(21) Appl. No.: 17/830,817

(22) Filed: Jun. 2, 2022

Related U.S. Application Data
(60) Provisional application No. 63/286,380, filed on Dec. 6, 2021.
(60) Provisional application No. 63/196,477, filed on Jun. 3, 2021.

(51) Int. Cl. C07K 16/40; A61P 35/00; C07K 16/30

(52) U.S. Cl. CPC C07K 16/40; A61P 35/00; C07K 16/30; A61K 2039/545

(57) ABSTRACT

The invention provides methods and uses of treating cancer with and IgG4 anti-CD39_229p antibody in combination with pembrolizumab, wherein the IgG4 anti-CD39_229p antibody is provided at particular doses and dosages.

### RELATED APPLICATIONS

This application claims priority to U.S. Provisional Patent Application Ser. Nos. 63/286,380, filed Dec. 6, 2021, and 63/196,477, filed Jun. 3, 2021, the entire disclosures of which are hereby incorporated herein by reference.

### SEQUENCE LISTING

The instant application contains a Sequence Listing which has been submitted electronically in ASCII format and is hereby incorporated by reference in its entirety. Said ASCII copy, created on Sep. 19, 2022, is named 728647_SRF9-001_ST25.txt and is 27,251 bytes in size.

### FIELD OF THE INVENTION

Methods of treating cancer with an anti-CD39 antibody at particular doses and dosages in combination with pembrolizumab are provided.

### BACKGROUND

The immune system acts through suppressive pathways to prevent cancerous cells from growing. Cancers use various mechanisms to subvert immune suppressive pathways in order to avoid recognition and elimination by immune cells, and to allow disease to progress. Immunotherapies fight cancer by modifying the patient's immune system by either directly stimulating rejection-type processes or by blocking suppressive pathways.

Adenosine is an immunomodulatory metabolite within the tumor microenvironment (TME) that interferes with the immune system's anti-tumor responses. In some cancers, extracellular adenosine accumulates and subsequently inhibits the function of immune cells, including T cells, dendritic cells (DC), and natural killer (NK) cells, thereby contributing to anti-tumor immune suppression and supporting tumor growth.

The ectonucleotidase CD39 hydrolyzes extracellular adenosine triphosphate (ATP) and adenosine diphosphate (ADP) to generate adenosine monophosphate (AMP), which is then converted to adenosine by CD73. Extracellular adenosine binds to adenosine receptors on immune cells, thereby suppressing the immune system. Overexpression of CD39 is associated with poor prognosis in patients with certain types of cancer. Within the TME, the adenosine pathway refers to the extracellular conversion of ATP to adenosine and the signaling of adenosine through the A2A/A2B adenosine receptors on immune cells. Under normal conditions, CD39 works to maintain the balance of extracellular levels of immunosuppressive adenosine and immunostimulatory ATP. In healthy tissues, ATP is barely detectable in the extracellular environment due to rapid breakdown by CD39 and conversion to adenosine by CD73. Under conditions of cellular stress, including cancer, extracellular ATP levels rise significantly leading to high levels of adenosine, which acts to suppress recognition of the tumor by the immune system and the anti-tumor response. While maintained levels of ATP increase T cell proliferation, dendritic cell maturation and pro-inflammatory cytokine levels, the accumulation of adenosine leads to immunosuppression. CD39 inhibition decreases immunosuppressive adenosine while stimulating immune responses by potentiating ATP levels in the TME.

There continues to be an unmet need for the development of methods of treating cancer. Novel combinations with existing therapies and therapeutic regimens are also needed to more effectively combat various cancers.

Provided herein are methods and uses for treating cancer comprising administering an IgG4 anti-CD39_229p antibody at particular doses and dosages, e.g., administered as a flat (or fixed) dose between 20 and 2000 mg at particular dosing intervals, in combination with pembrolizumab, an anti-PD-1 antibody. The methods and uses disclosed herein provide the benefit of treating various types of cancer, including relapsed or refractory solid tumors, while being well-tolerated.

### SUMMARY OF THE DISCLOSURE

Embodiment 1. A method of treating cancer in a human subject in need thereof comprising administering (i) a pharmaceutical composition comprising an IgG4 anti-CD39_229p antibody and (ii) a pharmaceutical composition comprising pembrolizumab or a pembrolizumab variant, wherein the IgG4 anti-CD39_229p antibody is administered at a dose of 20, 70, 200, 700, 1400, or 2000 mg.
Embodiment 2. The method of embodiment 1, wherein the IgG4 anti-CD39_229p antibody is administered intravenously.
Embodiment 3. The method of embodiment 1 or embodiment 2, wherein the IgG4 anti-CD39_229p antibody is administered once every 1, 2, 3, 4, 5 or 6 weeks.
Embodiment 4. The method of any one of embodiments 1-3, wherein the IgG4 anti-CD39_229p antibody is administered once every 2 weeks.
Embodiment 5. The method of any one of embodiments 1-4, wherein the pembrolizumab or pembrolizumab variant is administered at a dosage of 200 mg every 3 weeks.
Embodiment 6. The method of any one of embodiments 1-4, wherein the pembrolizumab or pembrolizumab variant is administered at a dosage of 400 mg every 6 weeks.
Embodiment 7. The method of any one of embodiments 1-6, wherein the pembrolizumab or pembrolizumab variant is administered intravenously.
Embodiment 8. A pharmaceutical composition comprising an IgG4 anti-CD39_229p antibody and a pharmaceutical composition comprising pembrolizumab or a pembrolizumab variant for use in treating cancer in a human subject in need thereof, wherein the IgG4 anti-CD39_229p antibody is administered at a dose of 20, 70, 200, 700, 1400, or 2000 mg.
Embodiment 9. The use of embodiment 8, wherein the IgG4 anti-CD39_229p antibody is administered intravenously.
Embodiment 10. The use of embodiment 8 or 9, wherein the IgG4 anti-CD39_229p antibody is administered once every 1, 2, 3, 4, 5 or 6 weeks.
Embodiment 11. The use of any one of embodiments 8-10, wherein the anti-CD39 antibody is administered once every two weeks.
Embodiment 12. The use of any one of embodiments 8-11, wherein the pembrolizumab or pembrolizumab variant is administered at a dosage of 200 mg every 3 weeks.
Embodiment 13. The use of any one of embodiments 8-11, wherein the pembrolizumab or pembrolizumab variant is administered at a dosage of 400 mg every 6 weeks.
Embodiment 14. The use of any one of embodiments 8-13, wherein the pembrolizumab or pembrolizumab variant is administered intravenously.
Embodiment 15. The method or use of any one of the preceding embodiments, wherein the cancer is newly diagnosed or non-metastatic.
Embodiment 16. The method or use of any one of the preceding embodiments, wherein the cancer is advanced.
Embodiment 17. The method or use of any one of the preceding embodiments, wherein the cancer is refractory.
Embodiment 18. The method or use of any one of the preceding embodiments, wherein the cancer is metastatic.
Embodiment 19. The method or use of any one of the preceding embodiments, wherein the cancer is a solid tumor.
Embodiment 20. The method or use of any one of the preceding embodiments, wherein the cancer is an advanced solid tumor.
Embodiment 21. The method or use of any one of the preceding embodiments, wherein the cancer is a relapsed solid tumor.
Embodiment 22. The method or use of any one of the preceding embodiments, wherein the cancer is a refractory solid tumor.
Embodiment 23. The method or use of any one of the preceding embodiments, wherein the cancer is a metastatic solid tumor.
Embodiment 24. The method or use of any one of the preceding embodiments, wherein the cancer is carcinoma, lymphoma, blastoma, sarcoma, or leukemia.
Embodiment 25. The method or use of any one of the preceding embodiments, wherein the cancer is pancreatic cancer.
Embodiment 26. The method or use of any one of the preceding embodiments, wherein the cancer is gastric cancer.
Embodiment 27. The method or use of any one of the preceding embodiments, wherein the cancer is prostate cancer.
Embodiment 28. The method or use of any one of the preceding embodiments, wherein the cancer is endometrial cancer.
Embodiment 29. The method or use of any one of the preceding embodiments, wherein the cancer is non-small cell lung cancer.
Embodiment 30. The method or use of any one of the preceding embodiments, wherein the cancer is colorectal cancer.
Embodiment 31. The method or use of any one of the preceding embodiments, wherein the cancer is ovarian cancer.
Embodiment 32. The method or use of any one of the preceding embodiments, wherein the cancer is squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, or various types of head and neck cancer (including squamous cell carcinoma of the head and neck).
Embodiment 33. The method or use of any one of the preceding embodiments, wherein the IgG4 anti-CD39_229p antibody and pembrolizumab or pembrolizumab variant are administered sequentially.
Embodiment 34. The method or use of embodiment 33, wherein the pembrolizumab or pembrolizumab variant is administered before the IgG4 anti-CD39_229p antibody is administered.
Embodiment 35. The method or use of any one of the preceding embodiments, wherein the IgG4 anti-CD39_229p antibody is administered at a dose of 20 mg intravenously every 2 weeks.
Embodiment 36. The method or use of any one of the preceding embodiments, wherein the IgG4 anti-CD39_229p antibody is administered at a dose of 70 mg intravenously every 2 weeks.
Embodiment 37. The method or use of any one of the preceding embodiments, wherein the IgG4 anti-CD39_229p antibody is administered at a dose of 200 mg intravenously every 2 weeks.
Embodiment 38. The method or use of any one of the preceding embodiments, wherein the IgG4 anti-CD39_229p antibody is administered at a dose of 700 mg intravenously every 2 weeks.
Embodiment 39. The method or use of any one of the preceding embodiments, wherein the IgG4 anti-CD39_229p antibody is administered at a dose of 1400 mg intravenously every 2 weeks.
Embodiment 40. The method or use of any one of the preceding embodiments, wherein the IgG4 anti-CD39_229p antibody is administered at a dose of 2000 mg intravenously every 2 weeks.
Embodiment 41. A method of treating cancer in a human subject in need thereof comprising administering a pharmaceutical composition comprising an IgG4 anti-CD39_229p antibody and a pharmaceutical composition comprising pembrolizumab or a pembrolizumab variant, wherein the IgG4 anti-CD39_229p antibody is administered at a dose of 20 mg intravenously every 2 weeks, and wherein pembrolizumab or the pembrolizumab variant is administered at a dose of 200 mg intravenously every 3 weeks. Embodiment 42. A method of treating cancer in a human subject in need thereof comprising administering a pharmaceutical composition comprising an IgG4 anti-CD39_229p antibody and a pharmaceutical composition comprising pembrolizumab or a pembrolizumab variant, wherein the IgG4 anti-CD39_229p antibody is administered at a dose of 70 mg intravenously every 2 weeks, and wherein pembrolizumab or the pembrolizumab variant is administered at a dose of 200 mg intravenously every 3 weeks. Embodiment 43. A method of treating cancer in a human subject in need thereof comprising administering a pharmaceutical composition comprising an IgG4 anti-CD39_229p antibody and a pharmaceutical composition comprising pembrolizumab or a pembrolizumab variant, wherein the IgG4 anti-CD39_229p antibody is administered at a dose of 200 mg intravenously every 2 weeks, and wherein pembrolizumab or the pembrolizumab variant is administered at a dose of 200 mg intravenously every 3 weeks.
Embodiment 44. A method of treating cancer in a human subject in need thereof comprising administering a pharmaceutical composition comprising an IgG4 anti-CD39_229p antibody and a pharmaceutical composition comprising pembrolizumab or a pembrolizumab variant, wherein the IgG4 anti-CD39_229p antibody is administered at a dose of 700 mg intravenously every 2 weeks, and wherein pembrolizumab or the pembrolizumab variant is administered at a dose of 200 mg intravenously every 3 weeks.
Embodiment 45. A method of treating cancer in a human subject in need thereof comprising administering a pharmaceutical composition comprising an IgG4 anti-CD39_229p antibody and a pharmaceutical composition comprising pembrolizumab or a pembrolizumab variant, wherein the IgG4 anti-CD39_229p antibody is administered at a dose of 1400 mg intravenously every 2 weeks, and wherein pembrolizumab or the pembrolizumab variant is administered at a dose of 200 mg intravenously every 3 weeks.
Embodiment 46. A method of treating cancer in a human subject in need thereof comprising administering a pharmaceutical composition comprising an IgG4 anti-CD39_229p antibody and a pharmaceutical composition comprising pembrolizumab or a pembrolizumab variant, wherein the IgG4 anti-CD39_229p antibody is administered at a dose of 2000 mg intravenously every 2 weeks, and wherein pembrolizumab or the pembrolizumab variant is administered at a dose of 200 mg intravenously every 3 weeks.
Embodiment 47. A method of treating cancer in a human subject in need thereof comprising administering a pharmaceutical composition comprising an IgG4 anti-CD39_229p antibody and a pharmaceutical composition comprising pembrolizumab or a pembrolizumab variant, wherein the IgG4 anti-CD39_229p antibody is administered at a dose of 20 mg intravenously every 2 weeks, and wherein pembrolizumab or the pembrolizumab variant is administered at a dose of 400 mg intravenously every 6 weeks.
Embodiment 48. A method of treating cancer in a human subject in need thereof comprising administering a pharmaceutical composition comprising an IgG4 anti-CD39_229p antibody and a pharmaceutical composition comprising pembrolizumab or a pembrolizumab variant, wherein the IgG4 anti-CD39_229p antibody is administered at a dose of 70 mg intravenously every 2 weeks, and wherein pembrolizumab or the pembrolizumab variant is administered at a dose of 400 mg intravenously every 6 weeks.
Embodiment 49. A method of treating cancer in a human subject in need thereof comprising administering a pharmaceutical composition comprising an IgG4 anti-CD39_229p antibody and a pharmaceutical composition comprising pembrolizumab or a pembrolizumab variant, wherein the IgG4 anti-CD39_229p antibody is administered at a dose of 200 mg intravenously every 2 weeks, and wherein pembrolizumab or the pembrolizumab variant is administered at a dose of 400 mg intravenously every 6 weeks.
Embodiment 50. A method of treating cancer in a human subject in need thereof comprising administering a pharmaceutical composition comprising an IgG4 anti-CD39_229p antibody and a pharmaceutical composition comprising pembrolizumab or a pembrolizumab variant, wherein the IgG4 anti-CD39_229p antibody is administered at a dose of 700 mg intravenously every 2 weeks, and wherein pembrolizumab or the pembrolizumab variant is administered at a dose of 400 mg intravenously every 6 weeks.
Embodiment 51. A method of treating cancer in a human subject in need thereof comprising administering a pharmaceutical composition comprising an IgG4 anti-CD39_229p antibody and a pharmaceutical composition comprising pembrolizumab or a pembrolizumab variant, wherein the IgG4 anti-CD39_229p antibody is administered at a dose of 1400 mg intravenously every 2 weeks, and wherein pembrolizumab or the pembrolizumab variant is administered at a dose of 400 mg intravenously every 6 weeks.
Embodiment 52. A method of treating cancer in a human subject in need thereof comprising administering a pharmaceutical composition comprising an IgG4 anti-CD39_229p antibody and a pharmaceutical composition comprising pembrolizumab or a pembrolizumab variant, wherein the IgG4 anti-CD39_229p antibody is administered at a dose of 2000 mg intravenously every 2 weeks, and wherein pembrolizumab or the pembrolizumab variant is administered at a dose of 400 mg intravenously every 6 weeks.

### BRIEF DESCRIPTION OF THE FIGURES

FIG. 1: Graphical depiction (interim results) of the percent target lesion change over time for each patient receiving clone 22 and pembrolizumab treatment. Three (3) patients were treated with 700 mg clone 22 (DL4: cross plot) and six (6) patients were treated with 1400 mg clone 22 (DL5: circle plot) in combination with pembrolizumab (200 mg). Patient responses were assessed in accordance with Response Evaluation Criteria in Solid Tumors (RECIST) v1.1.

FIG. 2: Graphical depiction (interim results) for the best percent change from baseline in sum of target lesions for each patient dosing group. Three (3) patients were treated with 700 mg clone 22 (DL4) and six (6) patients were treated with 1400 mg clone 22 (DL5) in combination with pembrolizumab (200 mg). Patient responses were assessed in accordance with Response Evaluation Criteria in Solid Tumors (RECIST) v1.1.

### DETAILED DESCRIPTION OF EMBODIMENTS OF THE INVENTION

### I. DEFINITIONS

In this application, the use of “or” means “and/or” unless stated otherwise. In the context of a multiple dependent claim, the use of “or” refers back to more than one preceding independent or dependent claim in the alternative only. The terms “comprising,” “including,” and “having” can be used interchangeably herein.

The term “CD39” refers to the ectonucleoside triphosphate diphospholydrolase 1 polypeptide encoded in humans by the ENTPD1 gene. Other names for CD39 include ENTPD1, E-NTPDase1, cluster of differentiation 39, ATPDase, NTPDase-1, and SPG64. CD39 catalyzes the hydrolysis of γ- and β-phosphate residues of extracellular nucleoside triphosphates (NTPs; e.g., adenosine triphosphate or ATP) and nucleoside diphosphates (NDPs; e.g., adenosine diphosphate or ADP), converting these molecules to the nucleoside monophosphate (NMP; e.g., adenosine monophosphate or AMP) derivative. An exemplary amino acid sequence of CD39 is at NCBI Reference Sequence: NP_001767.3.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells or leukemic cancer cells. The term “tumor” is used herein to refer to a cell or cells that comprise a cancer. The term “tumor growth” is used herein to refer to proliferation or growth by a cell or cells that comprise a cancer that leads to a corresponding increase in the size or extent of the cancer.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ϑ, γ, and μ, respectively.

Administration “in combination with” pembrolizumab includes simultaneous (concurrent) and consecutive (sequential) administration in any order.

The term “cytotoxic agent” as used herein refers to a substance that inhibits or prevents a cellular function and/or causes cell death or destruction. Cytotoxic agents include, but are not limited to, radioactive isotopes (e.g., At211, 1131, 1125, Y90, Re186, Re188, Sm153, Bi212, P32, Pb212 and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “dosage” refers to the amount and period of time of an administration. A “dose” refers to the amount administered. A “dosing interval” refers to the period of time between doses.

The term “Fc region” herein is used to define a C-terminal region of an immunoglobulin heavy chain that contains at least a portion of the constant region. The term includes native sequence Fc regions and variant Fc regions. In some embodiments, a human IgG heavy chain Fc region extends from Cys226, or from Pro230, to the carboxyl-terminus of the heavy chain. However, the C-terminal lysine (Lys447) of the Fc region may or may not be present (numbering in this paragraph is according to the EU numbering system, also called the EU index, as described in Kabat et al., Sequences of Proteins of Immunological Interest, 5^{th} Ed. Public Health Service, National Institutes of Health, Bethesda, Md., 1991).

“Framework,” “framework region,” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W. H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In some embodiments, for the VL, the subgroup is subgroup kappa I as in Kabat et al., supra. In some embodiments, for the VH, the subgroup is subgroup III as in Kabat et al., supra.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3).

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. In some embodiments, the human subject is referred to as a “patient.”

An increase in “interferon gamma response” or “IFNγ response” or “IFN-γ response” refers to any increase in activity associated with the cytokine IFNγ, and includes, e.g., an increase in IFNγ protein (e.g., present in the TME, secreted from a cell, or detected intracellularly), an increase in IFNγ gene expression in a cell (e.g., by measuring mRNA levels), an increase in gene expression of genes related to IFNγ (e.g., by measuring mRNA levels) (e.g., genes related to IFNγ include, e.g., Cc18, Apoe, C1qa, C1qb, C1qc, Arg1, Lgmn, Ms4a7, Lyz2, Cc17, Cd3g, Cxcr6, etc.). The increase may be detected in a sample from an individual after administration of a therapy e.g., as compared to a sample from an untreated individual. In some embodiments, increase may be detected in a sample from an individual after administration of a first and a second therapy e.g., as compared to a sample from an individual after administration of only the first or the second therapy.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“An IgG4 anti-CD39_229p antibody” as used herein refers to a full-length, fully human monoclonal IgG4 antibody with a proline in the heavy chain framework region that corresponds to position 229 of SEQ ID NO: 29, comprising a heavy chain variable region (VH) comprising the amino acid sequence of SEQ ID NO: 22 and a light chain variable region (VL) comprising the amino acid sequence of SEQ ID NO: 28. For clarity, clone 22 is an exemplary IgG4 anti-CD39_229p antibody. Additional antibodies may fall within the genus of IgG4 anti-CD39_229p antibodies so long as they are 1) IgG4; 2) comprise a heavy chain variable region (VH) comprising the amino acid sequence of SEQ ID NO: 22 and a light chain variable region (VL) comprising the amino acid sequence of SEQ ID NO: 28; and 3) comprise a proline in the heavy chain framework region that corresponds to position 229 of SEQ ID NO: 29.

The term “pharmaceutical formulation” or “pharmaceutical composition” or “pharmaceutically acceptable composition” refers to a preparation which is in such form as to permit the biological activity of an active ingredient contained therein to be effective, and which contains no additional components which are unacceptably toxic to a subject to which the formulation would be administered. Throughout the specification a reference to “an IgG4 anti-CD39_229p antibody” or “pembrolizumab,” is to be understood to also refer to a pharmaceutical formulation, composition, or pharmaceutically acceptable composition thereof. Thus, “an IgG4 anti-CD39_229p antibody” may refer to an IgG4 anti-CD39_229p antibody or a pharmaceutical formulation, composition, or pharmaceutically acceptable composition comprising an IgG4 anti-CD39_229p antibody, and “pembrolizumab” may refer to pembrolizumab or a pharmaceutical formulation, composition, or pharmaceutically acceptable composition comprising pembrolizumab.

A “pharmaceutically acceptable carrier” refers to an ingredient in a pharmaceutical formulation or composition, other than an active ingredient, which is nontoxic to a subject. A pharmaceutically acceptable carrier includes, but is not limited to, a buffer, excipient, stabilizer, and/or preservative.

The term “refractory,” as used herein, refers to a cancer that has not responded to a prior treatment. Refractory cancer includes a cancer that has exhibited an inadequate response to, or progressed on, a prior treatment, e.g., a prior treatment with an immuno-oncology or immunotherapy drug, e.g., with a blocking CTLA-4 or PD-1 antibody. In some embodiments, the cancer is refractory or resistant to a prior treatment, either intrinsically refractory or resistant (e.g., refractory to a PD-1 pathway antagonist), or a resistance or refractory state is acquired. The term “relapsed,” as used herein refers to a reoccurrence of a cancer in a subject. The term “metastatic,” as used herein refers to a cancer cell that has changed position from the place where it started, for example, the spread of a cancer from a primary site to another place in the body. The term “advanced,” as used herein refers to cancer that is unlikely to be cured or controlled with treatment.

As used herein, “treatment” (and grammatical variations thereof such as “treat” or “treating”) refers to clinical intervention in an attempt to alter the natural course of the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, disclosed antibodies are used to delay development of a disease or to slow the progression of a disease.

As used herein, “about” or “approximately” means an acceptable error for a particular value as determined by one of ordinary skill in the art, which depends in part on how the value is measured or determined.

As used herein, numeric ranges (e.g., 1-2000) are inclusive of the numbers defining the range. Also as used herein, measured and measurable values are understood to be approximate, taking into account significant digits and the error associated with the measurement.

### II. METHODS AND USES

Methods of treating cancer comprising administering an IgG4 anti-CD39_229p antibody at particular doses and dosages and pembrolizumab are provided. It is understood that the disclosed doses for an IgG4 anti-CD39_229p antibody include amounts that are approximately the amount of the disclosed doses. The doses disclosed for an IgG4 anti-CD39_229p antibody may be administered as a “flat” dose (or “fixed” dose) that does not depend on the weight of the subject.

In some embodiments, methods are provided for treating cancer in a human subject in need thereof comprising administering an IgG4 anti-CD39_229p antibody and pembrolizumab, wherein an IgG4 anti-CD39_229p antibody is administered at a fixed or flat dose. In some embodiments, methods are provided for treating cancer in a human subject in need thereof comprising administering an IgG4 anti-CD39_229p antibody and pembrolizumab, wherein an IgG4 anti-CD39_229p antibody is administered at a dose of 20, 70, 200, 700, 1400, or 2000 mg. In some embodiments, pembrolizumab is administered at a dose of 200 mg intravenously every 3 weeks. In some embodiments, pembrolizumab is administered at a dose of 400 mg intravenously every 6 weeks.

In some embodiments, methods are provided for treating cancer in a human subject in need thereof comprising administering an IgG4 anti-CD39_229p antibody and pembrolizumab, wherein the IgG4 anti-CD39_229p antibody is administered at a dose between 20-2000 mg, 70-2000 mg, 200-2000 mg, 700-2000 mg, or 1400-2000 mg. In some embodiments, the dose of the IgG4 anti-CD39_229p antibody is administered as a dosage once every 1, 2, 3, 4, 5 or 6 weeks. In some embodiments, the dose of an IgG4 anti-CD39_229p antibody is administered once every 2 weeks. In some embodiments, the dose of an IgG4 anti-CD39_229p antibody is administered intravenously. In some embodiments, pembrolizumab is administered at a dose of 200 mg intravenously every 3 weeks. In some embodiments, pembrolizumab is administered at a dose of 400 mg intravenously every 6 weeks.

In some embodiments, methods are provided for treating cancer in a human subject in need thereof comprising administering an IgG4 anti-CD39_229p antibody and pembrolizumab, wherein an IgG4 anti-CD39_229p antibody is administered at a dose between 20-2000 mg. In some embodiments, an IgG4 anti-CD39_229p antibody is administered once every 1, 2, 3, 4, 5 or 6 weeks. In some embodiments, an IgG4 anti-CD39_229p antibody is administered at a dose of 20 mg, once every 1 week. In some embodiments, an IgG4 anti-CD39_229p antibody is administered at a dose of 20 mg, once every 2 weeks. In some embodiments, an IgG4 anti-CD39_229p antibody is administered at a dose of 20 mg, once every 3 weeks. In some embodiments, an IgG4 anti-CD39_229p antibody is administered at a dose of 20 mg, once every 4 weeks. In some embodiments, an IgG4 anti-CD39_229p antibody is administered at a dose of 20 mg, once every 5 weeks. In some embodiments, an IgG4 anti-CD39_229p antibody is administered at a dose of 20 mg, once every 6 weeks. In some embodiments, an IgG4 anti-CD39_229p antibody is administered at a dose of 70 mg, once every 1 week. In some embodiments, an IgG4 anti-CD39_229p antibody is administered at a dose of 70 mg, once every 2 weeks. In some embodiments, an IgG4 anti-CD39_229p antibody is administered at a dose of 70 mg, once every 3 weeks. In some embodiments, an IgG4 anti-CD39_229p antibody is administered at a dose of 70 mg, once every 4 weeks. In some embodiments, an IgG4 anti-CD39_229p antibody is administered at a dose of 70 mg, once every 5 weeks. In some embodiments, an IgG4 anti-CD39_229p antibody is administered at a dose of 70 mg, once every 6 weeks. In some embodiments, an IgG4 anti-CD39_229p antibody is administered at a dose of 200 mg, once every 1 week. In some embodiments, an IgG4 anti-CD39_229p antibody is administered at a dose of 200 mg, once every 2 weeks. In some embodiments, an IgG4 anti-CD39_229p antibody is administered at a dose of 200 mg, once every 3 weeks. In some embodiments, an IgG4 anti-CD39_229p antibody is administered at a dose of 200 mg, once every 4 weeks. In some embodiments, an IgG4 anti-CD39_229p antibody is administered at a dose of 200 mg, once every 5 weeks. In some embodiments, an IgG4 anti-CD39_229p antibody is administered at a dose of 200 mg, once every 6 weeks. In some embodiments, an IgG4 anti-CD39_229p antibody is administered at a dose of 700 mg, once every 1 week. In some embodiments, an IgG4 anti-CD39_229p antibody is administered at a dose of 700 mg, once every 2 weeks. In some embodiments, an IgG4 anti-CD39_229p antibody is administered at a dose of 700 mg, once every 3 weeks. In some embodiments, an IgG4 anti-CD39_229p antibody is administered at a dose of 700 mg, once every 4 weeks. In some embodiments, an IgG4 anti-CD39_229p antibody is administered at a dose of 700 mg, once every 5 weeks. In some embodiments, an IgG4 anti-CD39_229p antibody is administered at a dose of 700 mg, once every 6 weeks. In some embodiments, an IgG4 anti-CD39_229p antibody is administered at a dose of 1400 mg, once every 1 week. In some embodiments, an IgG4 anti-CD39_229p antibody is administered at a dose of 1400 mg, once every 2 weeks. In some embodiments, an IgG4 anti-CD39_229p antibody is administered at a dose of 1400 mg, once every 3 weeks. In some embodiments, an IgG4 anti-CD39_229p antibody is administered at a dose of 1400 mg, once every 4 weeks. In some embodiments, an IgG4 anti-CD39_229p antibody is administered at a dose of 1400 mg, once every 5 weeks. In some embodiments, an IgG4 anti-CD39_229p antibody is administered at a dose of 1400 mg, once every 6 weeks. In some embodiments, an IgG4 anti-CD39_229p antibody is administered at a dose of 2000 mg, once every 1 week. In some embodiments, an IgG4 anti-CD39_229p antibody is administered at a dose of 2000 mg, once every 2 weeks. In some embodiments, an IgG4 anti-CD39_229p antibody is administered at a dose of 2000 mg, once every 3 weeks. In some embodiments, an IgG4 anti-CD39_229p antibody is administered at a dose of 2000 mg, once every 4 weeks. In some embodiments, an IgG4 anti-CD39_229p antibody is administered at a dose of 2000 mg, once every 5 weeks. In some embodiments, an IgG4 anti-CD39_229p antibody is administered at a dose of 2000 mg, once every 6 weeks. In some embodiments, the dose is administered intravenously. In some embodiments, pembrolizumab is administered at a dose of 200 mg intravenously every 3 weeks. In some embodiments, pembrolizumab is administered at a dose of 400 mg intravenously every 6 weeks.

In some embodiments, methods are provided for treating cancer in a human subject in need thereof comprising administering an IgG4 anti-CD39_229p antibody and pembrolizumab, wherein an IgG4 anti-CD39_229p antibody is administered at a dose between 20-2000 mg and pembrolizumab is administered at a dose of 200 mg every 3 weeks. In some embodiments, an IgG4 anti-CD39_229p antibody is administered every 1, 2, 3, 4, 5 or 6 weeks. In some embodiments, an IgG4 anti-CD39_229p antibody and pembrolizumab are administered to the subject by intravenous administration. In some embodiments, an IgG4 anti-CD39_229p antibody and pembrolizumab are administered sequentially. In some embodiments, pembrolizumab is administered before the anti-CD39 antibody is administered. In some embodiments, pembrolizumab is administered at least one hour before the anti-CD39 antibody is administered.

In some embodiments, methods of treating cancer in a human subject in need thereof are provided comprising administering an IgG4 anti-CD39_229p antibody and pembrolizumab, wherein an IgG4 anti-CD39_229p antibody is administered at a dose of 20 mg every 2 weeks. In some embodiments, the dosage is administered intravenously.

In some embodiments, methods of treating cancer in a human subject in need thereof are provided comprising administering an IgG4 anti-CD39_229p antibody and pembrolizumab, wherein an IgG4 anti-CD39_229p antibody is administered at a dose of 70 mg every 2 weeks. In some embodiments, the dosage is administered intravenously.

In some embodiments, methods of treating cancer in a human subject in need thereof are provided comprising administering an IgG4 anti-CD39_229p antibody and pembrolizumab, wherein an IgG4 anti-CD39_229p antibody is administered at a dose of 200 mg every 2 weeks. In some embodiments, the dosage is administered intravenously.

In some embodiments, methods of treating cancer in a human subject in need thereof are provided comprising administering an IgG4 anti-CD39_229p antibody and pembrolizumab, wherein an IgG4 anti-CD39_229p antibody is administered at a dose of 700 mg every 2 weeks. In some embodiments, the dosage is administered intravenously.

In some embodiments, methods of treating cancer in a human subject in need thereof are provided comprising administering an IgG4 anti-CD39_229p antibody and pembrolizumab, wherein an IgG4 anti-CD39_229p antibody is administered at a dose of 1400 mg every 2 weeks. In some embodiments, the dosage is administered intravenously.

In some embodiments, methods of treating cancer in a human subject in need thereof are provided comprising administering an IgG4 anti-CD39_229p antibody and pembrolizumab, wherein an IgG4 anti-CD39_229p antibody is administered at a dose of 2000 mg every 2 weeks. In some embodiments, the dosage is administered intravenously.

In some embodiments, methods of treating cancer in a human subject in need thereof are provided comprising administering an IgG4 anti-CD39_229p antibody and pembrolizumab, wherein the IgG4 anti-CD39_229p antibody is administered at a dose of 20 mg intravenously every 2 weeks, and wherein pembrolizumab is administered at a dose of 200 mg intravenously every 3 weeks or 400 mg intravenously every 6 weeks. In some embodiments, an IgG4 anti-CD39_229p antibody is administered at a dose of 70 mg intravenously every 2 weeks, and pembrolizumab is administered at a dose of 200 mg intravenously every 3 weeks. In some embodiments, an IgG4 anti-CD39_229p antibody is administered at a dose of 200 mg intravenously every 2 weeks, and pembrolizumab is administered at a dose of 200 mg intravenously every 3 weeks. In some embodiments, an IgG4 anti-CD39_229p antibody is administered at a dose of 700 mg intravenously every 2 weeks, and pembrolizumab is administered at a dose of 200 mg intravenously every 3 weeks. In some embodiments, an IgG4 anti-CD39_229p antibody is administered at a dose of 1400 mg intravenously every 2 weeks, and pembrolizumab is administered at a dose of 200 mg intravenously every 3 weeks. In some embodiments, an IgG4 anti-CD39_229p antibody is administered at a dose of 2000 mg intravenously every 2 weeks, and pembrolizumab is administered at a dose of 200 mg intravenously every 3 weeks.

In some embodiments, methods of treating cancer in a human subject in need thereof are provided comprising administering an IgG4 anti-CD39_229p antibody and pembrolizumab, wherein the IgG4 anti-CD39_229p antibody is administered at a dose of 20 mg intravenously every 2 weeks, and wherein pembrolizumab is administered at a dose of 400 mg intravenously every 6 weeks. In some embodiments, an IgG4 anti-CD39_229p antibody is administered at a dose of 70 mg intravenously every 2 weeks, and pembrolizumab is administered at a dose of 400 mg intravenously every 6 weeks. In some embodiments, an IgG4 anti-CD39_229p antibody is administered at a dose of 200 mg intravenously every 2 weeks, and pembrolizumab is administered at a dose of 400 mg intravenously every 6 weeks. In some embodiments, an IgG4 anti-CD39_229p antibody is administered at a dose of 700 mg intravenously every 2 weeks, and pembrolizumab is administered at a dose of 400 mg intravenously every 6 weeks. In some embodiments, an IgG4 anti-CD39_229p antibody is administered at a dose of 1400 mg intravenously every 2 weeks, and pembrolizumab is administered at a dose of 400 mg intravenously every 6 weeks. In some embodiments, an IgG4 anti-CD39_229p antibody is administered at a dose of 2000 mg intravenously every 2 weeks, and pembrolizumab is administered at a dose of 400 mg intravenously every 6 weeks.

In some embodiments, methods are provided for administering an IgG4 anti-CD39_229p antibody and pembrolizumab to a human subject in need thereof at particular doses and dosages wherein the administration results in enhancing, increasing and/or sustaining an anti-tumor immune response in the subject having a tumor. In some embodiments, the tumor is cancerous.

In further aspects, methods for treating cancer are provided where reducing or inhibiting the enzymatic activity of CD39 is desired comprising administering an IgG4 anti-CD39_229p antibody at particular doses and dosages in combination with pembrolizumab. In some embodiments, methods for enhancing, increasing and/or sustaining an anti-tumor immune response in a subject having a tumor are provided comprising administering an IgG4 anti-CD39_229p antibody at particular doses and dosages in combination with pembrolizumab. In some embodiments, the tumor is cancerous. In some embodiments, methods for treating cancer in a subject having cancer are provided comprising administering an IgG4 anti-CD39_229p antibody at particular doses and dosages in combination with pembrolizumab.

In some aspects, methods are provided for alleviating one or more symptoms of cancer in a subject comprising administering an IgG4 anti-CD39_229p antibody at particular doses and dosages in combination with pembrolizumab. In some aspects, methods are provided for reducing the number of symptoms or the severity of cancer in a subject comprising administering an IgG4 anti-CD39_229p antibody at particular doses and dosages in combination with pembrolizumab. In a particular embodiment, the symptom of the cancer is a tumor, and a reduction is a reduction in size of a tumor, the failure of the tumor to grow, or the elimination of the tumor.

In some embodiments, administration of an IgG4 anti-CD39_229p antibody and pembrolizumab may inhibit the growth of at least one tumor in the subject. In some embodiments, methods for inhibiting CD39 in a tissue of a subject having cancer are provided, comprising administering an IgG4 anti-CD39_229p antibody at particular doses and dosages in combination with pembrolizumab to the subject, wherein the administration reduces CD39 activity or total amount of CD39 in the tissue as compared to the activity or amount prior to administration. In some embodiments, methods of preventing CD39-mediated conversion of eATP and eADP to extracellular adenosine in a tissue of a subject having cancer are provided, comprising administering an IgG4 anti-CD39_229p antibody at particular doses and dosages in combination with pembrolizumab, wherein the administration reduces extracellular adenosine levels within the tumor microenvironment of the tissue. In some embodiments, methods of inhibiting CD39 activity in a tissue of a subject having cancer are provided, comprising administering an IgG4 anti-CD39_229p antibody and pembrolizumab, wherein the administration improves the ability to mount an immune response against a tumor cell.

In some embodiments, the methods for treating a human subject having cancer by administration of an IgG4 anti-CD39_229p antibody at particular doses and dosages and pembrolizumab result in infiltration of innate immune cells into the tumor microenvironment. In some embodiments, the infiltration of innate immune cells is greater in a sample from an individual after administration of a therapy e.g., as compared to a sample from an untreated individual. In some embodiments, the innate immune cells are myeloid cells. In some embodiments, the innate immune cells are tumor-associated macrophages. In some embodiments, the tumor-associated macrophages are positive for expression the F4/80 antigen. In some embodiments, the innate immune cells are NK cells.

In some embodiments, the disclosure provide an IgG4 anti-CD39_229p antibody at particular doses and dosages and pembrolizumab for use in treating cancer in a human subject in need thereof.

A. An IgG4 Anti-CD39_229 p Antibody

In some embodiments, an IgG4 anti-CD39_229p antibody in the disclosed methods and uses binds to human CD39. In some embodiments, an IgG4 anti-CD39_229p antibody in the disclosed methods and uses binds to and inhibits human CD39. In some embodiments, an IgG4 anti-CD39_229p antibody reduces or inhibits the enzymatic activity of human CD39. In some embodiments, an IgG4 anti-CD39_229p antibody binds to recombinant CD39 and/or to membrane bound human CD39.

In some embodiments, an IgG4 anti-CD39_229p antibody binds to human CD39 with an equilibrium dissociation constant (KD) of less than 10 nM. In some embodiments, an IgG4 anti-CD39_229p antibody binds to human CD39 with a K_{D} of about 1.11 nM. In some embodiments, an IgG4 anti-CD39_229p antibody binds to human CD39 and cynomolgus monkey CD39 but do not bind to mouse CD39 or rat CD39.

In some embodiments, the methods and uses provide that an IgG4 anti-CD39_229p antibody inhibits or reduces conversion by human CD39 of extracellular adenosine triphosphate (eATP) or extracellular adenosine diphosphate (eADP) to extracellular adenosine monophosphate (eAMP). In some embodiments, an IgG4 anti-CD39_229p antibody increases the amount of eATP. In some embodiments, an IgG4 anti-CD39_229p antibody reduces or decreases the amount of extracellular adenosine. In some embodiments, the methods and uses provide that an IgG4 anti-CD39_229p antibody maintains, increases or enhances an immunostimulatory level of eATP. In some embodiments, an IgG4 anti-CD39_229p antibody antagonizes human CD39 in a tumor microenvironment of a tissue. In some embodiments, the methods provide that an IgG4 anti-CD39_229p antibody cross-reacts with cynomolgus CD39. In some embodiments, the methods provide that an IgG4 anti-CD39_229p antibody increases or enhances proliferation of a lymphocyte. In some embodiments, the methods provide that an IgG4 anti-CD39_229p antibody increases or enhances macrophage infiltration in tumors. In some embodiments, the methods provide that an IgG4 anti-CD39_229p antibody binds to CD39 and inhibits CD39 within a normal or cancerous tissue. In some embodiments, the tissue is in the uterus, cervix, lung, prostate, breast, head, neck, colon, or ovary. In some embodiments, the tissue is in the uterus. In some embodiments, within the uterus, an IgG4 anti-CD39_229p antibody inhibits CD39 in the myometrium.

Table 4 below provides the sequences of clone 22, which is an exemplary antibody falling within the scope of an IgG4 anti-CD39_229p antibody.

In some embodiments, the administration of an IgG4 anti-CD39_229p antibody and pembrolizumab reduces CD39 activity or total amount of CD39 in the tissue as compared to the activity or amount prior to administration.

Clone 22, disclosed in Table 4 herein, is a fully human anti-CD39 monoclonal antibody that binds to human CD39 with nanomolar affinity and potently inhibits its enzymatic activity. Clone 22 prevents CD39-mediated conversion of ATP and adenosine diphosphate (ADP) to adenosine monophosphate (AMP) and phosphate, leading to a reduction in adenosine levels within the TME.

In certain embodiments, the IgG4 anti-CD39_229p antibody comprises a heavy chain (HC) comprising the amino acid sequence of the heavy chain of clone 22. In certain embodiments, the IgG4 anti-CD39_229p antibody comprises a light chain (LC) comprising the amino acid sequence of the heavy chain of clone 22. In some embodiments, the IgG4 anti-CD39_229p antibody comprises a heavy chain comprising the amino acid sequence of the heavy chain of antibody clone number 22 and a light chain comprising the light chain amino acid sequence of antibody clone number 22.

B. Pembrolizumab

“Pembrolizumab” (formerly known as MK-3475, SCH 900475 and lambrolizumab) alternatively referred to herein as “pembro,” is a humanized IgG4 mAb with the structure described in WHO Drug Information, Vol. 27, No. 2, pages 161-162 (2013) and which comprises the heavy and light chain amino acid sequences and CDRs described in Table 1. Pembrolizumab has been approved by the U.S. FDA as described in the Prescribing Information for KEYTRUDA™ (Merck & Co., Inc., Whitehouse Station, N.J., USA; initial U.S. approval 2014, updated March 2021).

“Pembrolizumab variant” as used herein means a monoclonal antibody that comprises heavy chain and light chain sequences that are identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g. the variant positions are located in the FR regions or the constant region, and optionally has a deletion of the C-terminal lysine residues of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

In some embodiments, of the methods of treating cancer or pharmaceutical compositions claimed herein, the dose of pembrolizumab is a fixed or flat dose. In some embodiments, of the methods of treating cancer or pharmaceutical compositions claimed herein, the dose of pembrolizumab is 200 mg. In some embodiments, the dosing interval of pembrolizumab is every 3 weeks. In some embodiments, the dosage of pembrolizumab is 200 mg every 3 weeks. In some embodiments, the dosing interval of pembrolizumab is every 6 weeks. In some embodiments, the dosage of pembrolizumab is 400 mg every 6 weeks.

In some embodiments, a composition comprising pembrolizumab may be provided as a liquid formulation or prepared by reconstituting a lyophilized powders with sterile water for injection prior to use. WO 2012/135408 describes the preparation of liquid and lyophilized medicaments comprising pembrolizumab that are suitable for use in the methods described herein. In some embodiments, a composition comprising pembrolizumab is provided in a glass vial which contains about 100 mg of pembrolizumab in 4 mL of solution. Each 1 mL of solution contains 25 mg of pembrolizumab and is formulated in: L-histidine (1.55 mg), polysorbate 80 (0.2 mg), sucrose (70 mg) and Water for Injection, USP. The solution requires dilution for IV infusion.

TABLE 1
Exemplary anti-human PD-1 antibodies
A. Comprises light and heavy chain CDRs of hPD-1.09A in WO2008/156712 (light and
heavy chain CDRs of pembrolizumab)

| | |
|---|---|
| CDRL1 | RASKGVSTSGYSYLH |
| | SEQ ID NO: 1 |
| CDRL2 | LASYLES |
| | SEQ ID NO: 2 |
| CDRL3 | QHSRDLPLT |
| | SEQ ID NO: 3 |
| CDRH1 | NYYMY |
| | SEQ ID NO: 6 |
| CDRH2 | GINPSNGGTNFNEKFKN |
| | SEQ ID NO: 7 |
| CDRH3 | RDYRFDMGFDY |
| | SEQ ID NO: 8 |

B. Comprises the mature h109A heavy chain variable (VH) region and one of the mature
K09A light chain variable (VL) regions in WO 2008/156712

| | |
|---|---|
| Heavy chain VH | QVQLVQSGVEVKKPGASVKVSCKASGYTFTNYYMYWVRQA |
| | PGQGLEWMGGINPSNGGTNFNEKFKNRVTLTTDSSTTTAYME |
| | LKSLQFDDTAVYYCARRDYRFDMGFDYWGQGTTVTVSS |
| | SEQ ID NO: 9 (VH of pembrolizumab) |
| Light chain VL | EIVLTQSPATLSLSPGERATLSCRASKGVSTSGYSYLHWYQQK |
| | PGQAPRLLIYLASYLESGVPARESGSGSGTDFTLTISSLEPEDFA |
| | VYYCQHSRDLPLTFGGGTKVEIK |
| | SEQ ID NO: 4 (VL of pembrolizumab) |
| Heavy chain | QVQLVQSGVEVKKPGASVKVSCKASGYTFTNYYMYWVRQA |
| | PGQGLEWMGGINPSNGGTNFNEKFKNRVTLTTDSSTTTAYME |
| | LKSLQFDDTAVYYCARRDYRFDMGFDYWGQGTTVTVSSAST |
| | KGPSVFPLAPCSRSTSESTAALGCLVKDYFPEPVTVSWNSGAL |
| | TSGVHTFPAVLQSSGLYSLSSVVTVPSSSLGTKTYTCNVDHKP |
| | SNTKVDKRVESKYGPPCPPCPAPEFLGGPSVFLFPPKPKDTLMI |
| | SRTPEVTCVVVDVSQEDPEVQFNWYVDGVEVHNAKTKPREE |
| | QFNSTYRVVSVLTVLHQDWLNGKEYKCKVSNKGLPSSIEKTIS |
| | KAKGQPREPQVYTLPPSQEEMTKNQVSLTCLVKGFYPSDIAVE |
| | WESNGQPENNYKTTPPVLDSDGSFFLYSRLTVDKSRWQEGNV |
| | FSCSVMHEALHNHYTQKSLSLSLGK |
| | SEQ ID NO: 10 (heavy chain of pembrolizumab) |
| Light chain | EIVLTQSPATLSLSPGERATLSCRASKGVSTSGYSYLHWYQQK |
| | PGQAPRLLIYLASYLESGVPARFSGSGSGTDFTLTISSLEPEDFA |
| | VYYCQHSRDLPLTFGGGTKVEIKRTVAAPSVFIFPPSDEQLKSG |
| | TASVVCLLNNFYPREAKVQWKVDNALQSGNSQESVTEQDSK |
| | DSTYSLSSTLTLSKADYEKHKVYACEVTHQGLSSPVTKSFNRG |
| | EC |
| | SEQ ID NO: 5 (light chain of pembrolizumab) |

C. Recombinant Methods

Antibodies may be produced using recombinant methods and compositions, e.g., as described in U.S. Pat. No. 4,816,567, or as described in WO2019178269. In some embodiments, an isolated nucleic acid(s) encoding an anti-CD39 antibody described herein is provided. Such nucleic acid(s) may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid(s) are provided. In a further embodiment, a host cell comprising such nucleic acid(s) is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In some embodiments, the host cell is eukaryotic, e.g., a Chinese Hamster Ovary (CHO) cell or lymphoid cell (e.g., Y0, NS0, Sp20 cell). In some embodiments, a method of making an anti-CD39 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD39 antibody, a nucleic acid(s) encoding an antibody, e.g., as described above, is isolated and inserted into one or more vectors for further cloning and/or expression in a host cell. Such nucleic acid(s) may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

D. Treatment Measures

In some embodiments, results of the treatment methods and uses provided herein are evaluated. In some embodiments, disease progression of the subject having cancer may evaluated using known response evaluation criteria (e.g., Response Evaluation Criteria in Solid Tumors (RECIST)). In some embodiments, response to treatment is evaluated using tumor biopsies. In some embodiments, response to treatment is evaluated using a CT scan of chest, abdomen, pelvis, fluorodeoxyglucose-positron emission tomography, and/or magnetic resonance imaging. In some embodiments, response to treatment is evaluated by measuring antidrug antibodies, electrocardiograms (ECGs), and/or safety laboratory values. In some embodiments, response to treatment is evaluated by measuring levels of anti-CD39 antibody target occupancy and/or serum concentrations of anti-CD39 antibody. In some embodiments, response to treatment is evaluated by measuring levels of intratumoral CD39 enzymatic activity. In some embodiments, the objective response rate (ORR), duration of response (DoR), disease control rate (DCR), progression-free survival (PFS), and landmark PFS rate are determined. In some embodiments, response to treatment is evaluated by measuring changes in selected blood, serum/plasma, cytokines, and/or tumor tissue biomarkers, which may include gene and protein expression levels, tumor DNA mutation and copy number variations, and immune cell population subset enumeration and evaluation. In some embodiments, response to treatment is evaluated by measuring germline DNA polymorphic sequence variations in relation to the PK, pharmacodynamics, safety, and/or preliminary efficacy of the anti-CD39 antibody. In some embodiments, response to treatment is evaluated by measuring serum concentrations of the combination therapy agents.

E. Diseases and Disorders

In the methods and uses for treating cancer provided herein, cancers can be cancers with solid tumors or blood malignancies (e.g., liquid tumors). In some embodiments, the cancer is newly diagnosed. In some embodiments, the cancer is non-metastatic. In some embodiments, the cancer is advanced. In some embodiments, the cancer is relapsed. In some embodiments, the cancer is refractory. In some embodiments, the cancer is metastatic. In some embodiments, the cancer is a solid tumor. In some embodiments, the cancer is an advanced solid tumor. In some embodiments, the cancer is a relapsed solid tumor. In some embodiments the cancer is a refractory solid tumor. In some embodiments, the cancer is a metastatic solid tumor. In some embodiments, the cancer is an advanced, relapsed solid tumor. In some embodiments, the cancer is an advanced, refractory solid tumor. In some embodiments, the cancer is an advanced, metastatic solid tumor. In some embodiments, the cancer is a relapsed, refractory solid tumor. In some embodiments, the cancer is a relapsed, metastatic solid tumor. In some embodiments, the cancer is a refractory, metastatic tumor. In some embodiments, the tumor is an advanced, relapsed, refractory solid tumor. In some embodiments, the cancer is an advanced, relapsed, metastatic tumor. In some embodiments, the cancer is an advanced, refractory, metastatic tumor. In some embodiments, the cancer is a relapsed, refractory, metastatic solid tumor. In some embodiments, the tumor is an advanced, relapsed, refractory, metastatic solid tumor.

Non-limiting examples of cancers for treatment include squamous cell carcinoma, small-cell lung cancer, non-small cell lung cancer, squamous non-small cell lung cancer (NSCLC), nonsquamous NSCLC, glioma, gastrointestinal cancer, renal cancer (e.g., clear cell carcinoma), ovarian cancer, liver cancer, hepatocellular carcinoma (HCC), colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, sarcoma, sinonasal natural killer, melanoma (e.g., malignant melanoma, such as cutaneous or intraocular malignant melanoma and metastatic malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, angiosarcoma, spinal axis tumor, brain cancer, brain stem glioma, pituitary adenoma, Kaposi's sarcoma, epidermoid cancer, squamous cell cancer, T cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CIVIL), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B cell lymphomas, T cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T cell lymphoma/leukemia, mantle cell lymphoma, angio immunoblastic T cell lymphoma, angiocentric lymphoma, intestinal T cell lymphoma, primary mediastinal B-cell lymphoma, precursor T-lymphoblastic lymphoma, T-lymphoblastic; and lymphoma/leukaemia (T-Lbly/T-ALL), peripheral T cell lymphoma, lymphoblastic lymphoma, post-transplantation lymphoproliferative disorder, true histiocytic lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous system, including astrocytoma, oligodendroglioma, medulloblastoma, peripheral nerve sheath tumors, PNET, schwannomas; tumors of mesenchymal origin, including fibrosarcoma, rhabdomyoscaroma, and osteosarcoma; and other tumors, including xeroderma pigmentosum, keratoacanthoma, seminoma, thyroid follicular cancer and teratocarcinoma, hematopoietic tumors of lymphoid lineage, for example T cell and B cell tumors, including but not limited to T cell disorders such as T-prolymphocytic leukemia (T-PLL), including of the small cell and cerebriform cell type; large granular lymphocyte leukemia (LGL) of the T cell type; a/d T-NHL hepatosplenic lymphoma; peripheral/post-thymic T cell lymphoma (pleomorphic and immunoblastic subtypes); angiocentric (nasal) T cell lymphoma; cancer of the head or neck, renal cancer, rectal cancer, cancer of the thyroid gland; acute myeloid lymphoma, as well as any combinations of said cancers. The methods described herein can be used for treatment of metastatic cancers, and/or unresectable cancers, and/or relapsed cancers, and/or refractory cancers, and/or advanced cancers, and/or recurrent cancers. The methods described herein can be used for treatment of pancreatic cancer. The methods described herein can be used for treatment of gastric cancer. The methods described herein can be used for treatment of prostate cancer. The methods described herein can be used for treatment of endometrial cancer. The methods described herein can be used for treatment of non-small cell lung cancer. The methods described herein can be used for treatment of colorectal cancer. The methods described herein can be used for the treatment of ovarian cancer. The methods described herein can be used for the treatment of breast cancer.

In certain embodiments, an antibody described herein is administered to subjects having a cancer that has exhibited an inadequate response to, or progressed on, a prior treatment, e.g., a prior treatment with an immuno-oncology or immunotherapy drug. In some embodiments, the cancer is refractory or resistant to a prior treatment, either intrinsically refractory or resistant (e.g., refractory to a PD-1 pathway antagonist or other immune checkpoint therapy including CTLA4 inhibitors), or a resistance or refractory state is acquired. For example, an antibody described herein may be administered to subjects who are not responsive or not sufficiently responsive to a first therapy or who have disease progression following treatment, e.g., immune checkpoint therapies including anti-PD-1 pathway antagonist treatment or CTLA4 inhibitors, either alone or in combination with another therapy (e.g., with an anti-PD-1 pathway antagonist therapy). In other embodiments, an antibody described herein is administered to subjects who have not previously received (i.e., been treated with) an immuno-oncology agent, e.g., a PD-1 pathway antagonist.

### III. EXAMPLES

### Example 1. Clone 22 and Pembrolizumab Dose Escalation, Safety, and Tumor Biopsy Expansion Study in Patients with Advanced Solid Tumors

Patients with advanced solid tumors refractory to standard therapy were enrolled in a phase 1 study of clone 22 administered intravenously every 2 weeks and pembrolizumab administered every 3 weeks intravenously (NCT04336098) to establish the preliminary safety of the combination therapy and identify a schedule suitable for safety expansion. Clinical outcomes based on Response Evaluation Criteria in Solid Tumors, and pharmacokinetic (PK) and pharmacodynamic (PD) analyses were evaluated.

### Study Design:

The study was designed as a phase 1 open-label, first-in-human, combination therapy dose escalation, safety, and tumor biopsy expansion study in patients with advanced solid tumors. The study design includes a monotherapy dose escalation portion and a combination therapy dose escalation portion.

The doses for the monotherapy dose escalation portion of the study are shown in Table 2. The starting dose was 20 mg given intravenously (IV) once every 2 weeks.

TABLE 2
Dose Levels

| Dose Level | Clone 22 Dose | Number of Patients |
|---|---|---|
| 1 (starting dose) | 20 mg | N = 1-6 |
| 2 | 70 mg | N = 1-6 |
| 3 | 200 mg | N = 3-6 |
| 4 | 700 mg | N = 3-6 |
| 5 | 1400 mg | N = 3-6 |
| 6 | 2000 mg | N = 3-6 |

Note:
Escalation of dose levels may continue after completion of Dose Level 6 at the recommendation of the Safety Review Committee.

The combination therapy dose escalation study will enroll approximately 12 patients and was designed to evaluate the safety, tolerability, PK, and preliminary efficacy of clone 22 in combination with pembrolizumab in patients with locally advanced or metastatic solid tumors. Once the recommended Phase 2 dose is established based on the monotherapy study, up to 10 additional patients may be enrolled in the combination study.

Under the design, clone 22+pembrolizumab combination therapy cohorts receive a clone 22 monotherapy dose level that is no higher than the most-recently cleared dose level for clone 22 monotherapy. Pembrolizumab will be administered at a dose of 200 mg every 3 weeks. Patients will receive pembrolizumab intravenously (IV) over 30 minutes at a dose of 200 mg every 3 weeks, on Day 1 and Day 22 of each 6-week treatment cycle after all procedures and assessments have been completed. When scheduled on the same day, pembrolizumab is administered before the clone 22 dose, with clone 22 administered after a 1-hour observation period.

The primary study endpoint is dose limiting toxicities (DLTs), including e.g., hematologic toxicities (e.g., decreased neutrophil count, febrile neutropenia, decreased platelet count, and anemia), and nonhematologic toxicities (e.g., nausea, vomiting, diarrhea, fatigue, and immune mediated reactions). Secondary endpoints include adverse event (AEs), antidrug antibodies, electrocardiograms, safety laboratory values, serum concentrations of clone 22, levels of clone 22 target occupancy, objective response rate (ORR), duration of response, disease control rate, progression-free survival (PFS), landmark PFS rate, and levels of intratumoral CD39 enzymatic activity (in patients receiving pretreatment and on-treatment tumor biopsies).

Samples will be collected for biomarker analysis to investigate the biological effects of clone 22 at the molecular and cellular level, as well as to evaluate how changes in the markers and immune cell populations may relate to exposure and clinical outcomes. The goal of the biomarker assessments is to provide supportive data for the clinical study. There may be circumstances when a decision is made to stop a collection, not perform, or discontinue an analysis because of either practical or strategic reasons, e.g., inadequate sample number, sample quality issues precluding analysis. Therefore, sample collection and/or analysis may be omitted at the discretion of the study sponsor.

Archival tumor tissue/fresh tumor biopsy tissues samples may be analyzed for biomarkers, including in situ examination of markers on tumor cells, distinct immune cell populations, and other nontumor cellular compartments (e.g., stroma) by immunohistochemistry which may include, but are not limited to, CD39, PD-L1, and T-cell and macrophage populations as assessed by various immune cell markers (eg, CD8 and CD68).

### Interim Pembrolizumab Combination Results:

Three (3) patients were dosed with clone 22 (700 mg intravenously once every two weeks) in combination with pembrolizumab (200 mg administered as an IV infusion on Days 1 and 22 of a 6-week treatment cycle). There were no early safety signals.

### Updated Interim Pembrolizumab Combination Results:

Nine (9) patients were dosed with clone 22 (three (3) patients with 700 mg intravenously once every two weeks and six (6) patients with 1400 mg intravenously once every two weeks) in combination with pembrolizumab (200 mg administered as an IV infusion on Days 1 and 22 of a 6-week treatment cycle). Mean time on study as of the database snapshot date was 13.9 weeks and median time on study was 15 weeks. Patient demographics and baseline characteristics are summarized in Table 3 below.

TABLE 3
Patient demographics

| | |
|---|---|
| Median age, years (range) | 57 (42, 77) |
| Sex, n (%) | |
| Male | 3 (33%) |
| Female | 6 (67%) |
| ECOG PS at baseline, n (%) | |
| 0 | 6 (67%) |
| 1 | 3 (33%) |
| Median time since initial diagnosis, months (range) | 34 (17, 133) |
| Number of prior systemic therapies, n (%) | |
| 0 | 0 (0%) |
| 1-3 | 6 (67%) |
| 4-5 | 1 (11%) |
| ≥5 | 2 (22%) |
| Prior anti-PD-1/anti-PD-L1, n (%) | |
| Yes | 2 (22%) |
| No | 7 (78%) |

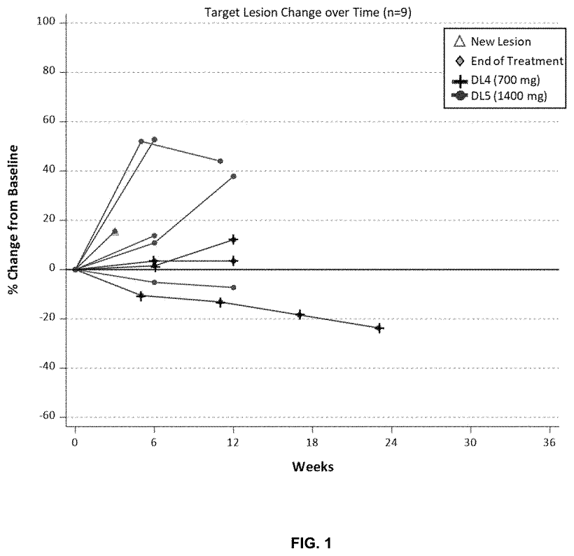

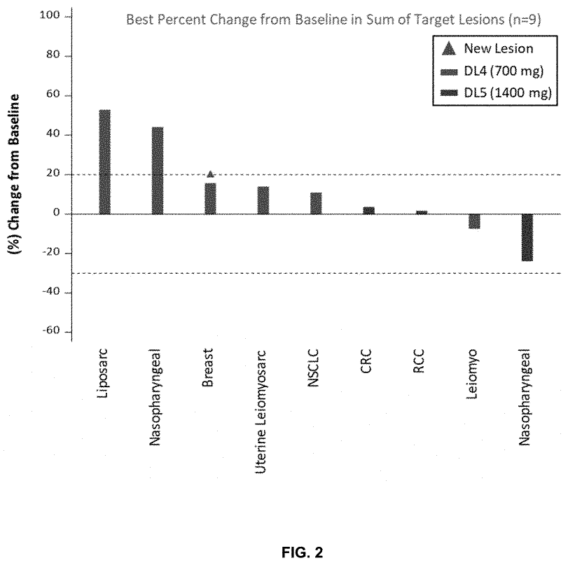

Patients with the following solid tumor types were enrolled in the study: liposarcoma (1), nasopharyngeal (2), breast (1), uterine leiomyosarcoma (1), non-small-cell lung (1), colorectal (1), renal cell (1), and leiomyosarcoma (1). Investigators assessed patient responses to treatment with clone 22 in combination with pembrolizumab in accordance with Response Evaluation Criteria in Solid Tumors (RECIST) v1.1. A graphical depiction of the interim results for percent change from baseline of the target lesion for each patient is shown in FIG. 1. A graphical depiction of interim results for the best percent change from baseline in the sum of target lesions for each patient dosing group is shown in FIG. 2.

Four (4) patients (44.4%) were evaluated to have stable disease and five (5) patients (55.6%) were evaluated to have progressive disease. Four (4) of eight (8) evaluable patients (50%) had disease stabilization at six weeks, with three (3) of the eight (8) exhibiting disease control at 12 weeks and one beyond 20 weeks. (see FIG. 1).

The most common TEAEs were pruritus (33%), diarrhea, myalgia, anemia, cough, and dizziness (22%, each). No dose-limiting toxicities have been observed. PK are linear and correlate strongly with PD measures of target occupancy.

### Example 2. Clone 22, Gemcitabine, Albumin-Bound Paclitaxel, and Pembrolizumab First Line Pancreatic Cancer Safety Study

Patients with pancreatic cancer were enrolled in a phase 1 study of clone 22 administered 1400 mg intravenously every 2 weeks, in combination with gemcitabine (1000 mg/m² administered as an IV infusion on Days 1, 8, and 15 of a 28-day cycle), albumin-bound paclitaxel (Abraxane) (125 mg/m² administered as an IV infusion over 30 to 40 minutes prior to gemcitabine on days 1, 8, and 15 of a 28-day cycle) and pembrolizumab (200 mg administered every 3 weeks intravenously) to establish the preliminary safety of the quadruple combination therapy. Clinical outcomes based on Response Evaluation Criteria in Solid Tumors (RECIST), and pharmacokinetic (PK) and pharmacodynamic (PD) analyses were evaluated.

### Example 3. Clone 22 and Pembrolizumab in PD-(L)1 Relapsed/Refractory Gastric Cancer, Gastroesophageal Junction Adenocarcinoma, and Non-Small-Cell Lung Cancer Safety Study

Patients with relapsed or refractory gastric cancer, gastroesophageal junction adenocarcinoma, and non-small-cell lung cancer were enrolled in a phase 1 study of clone 22 administered 1400 mg intravenously every 2 weeks in combination with pembrolizumab (200 mg administered every 3 weeks intravenously) to establish the preliminary safety of the combination therapy. Clinical outcomes based on Response Evaluation Criteria in Solid Tumors (RECIST), and pharmacokinetic (PK) and pharmacodynamic (PD) analyses were evaluated.

### Example 4. Clone 22 and Pembrolizumab in PD-(L)1 Treatment Naïve Gastric Cancer and Gastroesophageal Junction Adenocarcinoma Safety Study

Patients with PD-(L)1 treatment naïve gastric cancer and gastroesophageal junction adenocarcinoma were enrolled in a phase 1 study of clone 22 administered 1400 mg intravenously every 2 weeks in combination with pembrolizumab (200 mg administered every 3 weeks intravenously) to establish the preliminary safety of the combination therapy. Clinical outcomes based on Response Evaluation Criteria in Solid Tumors (RECIST), and pharmacokinetic (PK) and pharmacodynamic (PD) analyses were evaluated.

Although the foregoing invention has been described in some detail by way of illustration and example for purposes of clarity of understanding, the descriptions and examples should not be construed as limiting the scope of the disclosure. The disclosures of all patent and scientific literature cited herein are expressly incorporated in their entirety by reference.

TABLE 4

| SEQ | Clone | | |
|---|---|---|---|
| ID NO | No | Description | Sequence |
| 11 | 22 | VH CDR1 | GTFSSEGIS |
| 12 | 22 | VH CDR2 | SILPIFGTANYAQKFQG |
| 13 | 22 | VH CDR3 | AREAGYYRYRYFDL |
| 14 | 22 | VL CDR1 | RASQSVSSNLA |
| 15 | 22 | VL CDR2 | GASTRAT |
| 16 | 22 | VL CDR3 | QQHALWPLT |
| 17 | 22 | VH FR1 | QVQLVQSGAEVKKPGSSVKVSCKASG |
| 18 | 22 | VH FR2 | WVRQAPGQGLEWMG |
| 19 | 22 | VH FR3 | RVTITADESTSTAYMELSSLRSEDTAVYYC |
| 20 | 22 | VH FR4 | WGRGTLVTVSS |
| 21 | 22 | VH DNA | CAAGTGCAGTTGGTGCAGTCCGGAGCCGAAGTCAAGAAGCCCGG |
| | | | GTCGAGCGTGAAAGTGTCCTGCAAGGCTTCTGGTGGAACCTTCTC |
| | | | AAGCGAAGGGATCAGCTGGGTCAGACAGGCGCCGGGCCAGGGTC |
| | | | TGGAGTGGATGGGTTCCATTCTCCCGATCTTCGGAACCGCCAATT |
| | | | ACGCCCAGAAGTTCCAGGGTCGCGTGACCATCACCGCCGACGAA |
| | | | AGCACCTCGACGGCCTATATGGAATTGTCGTCCCTGCGGTCGGAA |
| | | | GATACAGCGGTGTACTACTGTGCGCGGGAAGCCGGGTACTACCG |
| | | | CTACCGCTACTTCGATCTGTGGGGAAGGGGAACTCTCGTGACTGT |
| | | | GTCGAGCG |
| 22 | 22 | VH Protein | QVQLVQSGAEVKKPGSSVKVSCKASGGTFSSEGISWVRQAPGQGLE |
| | | | WMGSILPIFGTANYAQKFQGRVTITADESTSTAYMELSSLRSEDTAV |
| | | | YYCAREAGYYRYRYFDLWGRGTLVTVSS |
| 23 | 22 | VL FR1 | EIVMTQSPATLSVSPGERATLSC |
| 24 | 22 | VL FR2 | WYQQKPGQAPRLLIY |
| 25 | 22 | VL FR3 | GIPARFSGSGSGTEFTLTISSLQSEDFAVYYC |
| 26 | 22 | VL FR4 | FGGGTKVEIK |
| 27 | 22 | VL DNA | GAAATAGTGATGACGCAGTCTCCAGCCACCCTGTCTGTGTCTCCA |
| | | | GGGGAAAGAGCCACCCTCTCCTGCAGGGCCAGTCAGAGTGTTAG |
| | | | CAGCAACTTAGCCTGGTACCAGCAGAAACCTGGCCAGGCTCCCA |
| | | | GGCTCCTCATCTATGGTGCATCCACCAGGGCCACTGGTATCCCAG |
| | | | CCAGGTTCAGTGGCAGTGGGTCTGGGACAGAGTTCACTCTCACCA |
| | | | TCAGCAGCCTGCAGTCTGAAGATTTTGCAGTTTATTACTGTCAGC |
| | | | AGCACGCCCTCTGGCCTCTCACTTTTGGCGGAGGGACCAAGGTTG |
| | | | AGATCAAA |
| 28 | 22 | VL Protein | EIVMTQSPATLSVSPGERATLSCRASQSVSSNLAWYQQKPGQAPRLL |
| | | | IYGASTRATGIPARFSGSGSGTEFTLTISSLQSEDFAVYYCQQHALWP |
| | | | LTFGGGTKVEIK |
| 29 | 22 | Heavy Chain | QVQLVQSGAEVKKPGSSVKVSCKASGGTFSSEGISWVRQAPGQGLE |
| | | Protein | WMGSILPIFGTANYAQKFQGRVTITADESTSTAYMELSSLRSEDTAV |
| | | | YYCAREAGYYRYRYFDLWGRGTLVTVSSASTKGPSVFPLAPCSRST |
| | | | SESTAALGCLVKDYFPEPVTVSWNSGALTSGVHTFPAVLQSSGLYSL |
| | | | SSVVTVPSSSLGTKTYTCNVDHKPSNTKVDKRVESKYGPPCPPCPAP |
| | | | EFLGGPSVFLFPPKPKDTLMISRTPEVTCVVVDVSQEDPEVQFNWYV |
| | | | DGVEVHNAKTKPREEQFNSTYRVVSVLTVLHQDWLNGKEYKCKVS |
| | | | NKGLPSSIEKTISKAKGQPREPQVYTLPPSQEEMTKNQVSLTCLVKGF |
| | | | YPSDIAVEWESNGQPENNYKTTPPVLDSDGSFFLYSRLTVDKSRWQE |
| | | | GNVFSCSVMHEALHNHYTQKSLSLSLG |
| 30 | 22 | Heavy Chain | CAAGTGCAGTTGGTGCAGTCCGGAGCCGAAGTCAAGAAGCCCGG |
| | | DNA | GTCGAGCGTGAAAGTGTCCTGCAAGGCTTCTGGTGGAACCTTCTC |
| | | | AAGCGAAGGGATCAGCTGGGTCAGACAGGCGCCGGGCCAGGGTC |
| | | | TGGAGTGGATGGGTTCCATTCTCCCGATCTTCGGAACCGCCAATT |
| | | | ACGCCCAGAAGTTCCAGGGTCGCGTGACCATCACCGCCGACGAA |
| | | | AGCACCTCGACGGCCTATATGGAATTGTCGTCCCTGCGGTCGGAA |
| | | | GATACAGCGGTGTACTACTGTGCGCGGGAAGCCGGGTACTACCG |
| | | | CTACCGCTACTTCGATCTGTGGGGAAGGGGAACTCTCGTGACTGT |
| | | | GTCGAGCGCCAGCACCAAGGGACCCAGCGTGTTCCCGCTGGCCC |
| | | | CTTGTTCACGATCCACTTCCGAAAGCACCGCTGCCCTTGGCTGCC |
| | | | TTGTCAAGGACTACTTCCCTGAGCCCGTCACTGTGTCGTGGAACA |
| | | | GCGGAGCTCTGACCTCCGGCGTCCACACCTTCCCGGCTGTGCTCC |
| | | | AGTCCTCCGGCCTGTACTCACTGTCCTCGGTGGTCACCGTGCCCT |
| | | | CCTCCTCCCTCGGTACCAAGACTTATACCTGCAACGTGGACCACA |
| | | | AGCCCTCCAACACCAAAGTGGATAAGAGAGTGGAGAGCAAATAC |
| | | | GGACCTCCCTGCCCTCCTTGCCCTGCGCCTGAGTTTCTGGGCGGA |
| | | | CCATCCGTCTTTCTGTTCCCACCGAAGCCCAAGGACACCCTCATG |
| | | | ATCTCCCGGACCCCCGAAGTGACCTGTGTGGTGGTGGACGTGTCA |
| | | | CAGGAGGACCCTGAAGTGCAGTTTAATTGGTACGTCGACGGCGT |
| | | | GGAAGTGCATAACGCAAAGACCAAGCCGCGGGAGGAACAGTTCA |
| | | | ACTCAACCTACCGCGTGGTGTCCGTGCTGACTGTGCTGCACCAGG |
| | | | ACTGGCTGAACGGAAAGGAGTATAAGTGCAAAGTCTCCAACAAG |
| | | | GGACTGCCGAGCAGCATCGAGAAAACCATTTCAAAAGCCAAGGG |
| | | | CCAGCCGAGAGAGCCCCAAGTGTACACTCTGCCGCCGAGCCAAG |
| | | | AGGAAATGACCAAGAACCAAGTGTCCCTCACTTGCCTGGTCAAG |
| | | | GGCTTCTACCCGTCGGACATCGCCGTGGAGTGGGAAAGCAACGG |
| | | | CCAGCCGGAAAACAACTACAAGACTACCCCTCCCGTCCTCGACTC |
| | | | CGACGGGTCCTTCTTCCTCTACTCCCGGCTGACTGTGGATAAGTC |
| | | | ACGGTGGCAGGAGGGAAACGTGTTCTCGTGCTCCGTGATGCACG |
| | | | AAGCCCTGCACAACCACTACACGCAGAAGTCCCTGTCCTTGTCCC |
| | | | TGGGG |
| 31 | 22 | Light Chain | EIVMTQSPATLSVSPGERATLSCRASQSVSSNLAWYQQKPGQAPRLL |
| | | Protein | IYGASTRATGIPARFSGSGSGTEFTLTISSLQSEDFAVYYCQQHALWP |
| | | | LTFGGGTKVEIKRTVAAPSVFIFPPSDEQLKSGTASVVCLLNNFYPRE |
| | | | AKVQWKVDNALQSGNSQESVTEQDSKDSTYSLSSTLTLSKADYEKH |
| | | | KVYACEVTHQGLSSPVTKSFNRGEC |
| 32 | 22 | Light Chain | GAAATAGTGATGACGCAGTCTCCAGCCACCCTGTCTGTGTCTCCA |
| | | DNA | GGGGAAAGAGCCACCCTCTCCTGCAGGGCCAGTCAGAGTGTTAG |
| | | | CAGCAACTTAGCCTGGTACCAGCAGAAACCTGGCCAGGCTCCCA |
| | | | GGCTCCTCATCTATGGTGCATCCACCAGGGCCACTGGTATCCCAG |
| | | | CCAGGTTCAGTGGCAGTGGGTCTGGGACAGAGTTCACTCTCACCA |
| | | | TCAGCAGCCTGCAGTCTGAAGATTTTGCAGTTTATTACTGTCAGC |
| | | | AGCACGCCCTCTGGCCTCTCACTTTTGGCGGAGGGACCAAGGTTG |
| | | | AGATCAAACGTACGGTGGCCGCTCCCTCCGTGTTCATCTTCCCAC |
| | | | CCTCCGACGAGCAGCTGAAGTCCGGCACCGCCTCCGTCGTGTGCC |
| | | | TGCTGAACAACTTCTACCCTCGCGAGGCCAAAGTGCAGTGGAAA |
| | | | GTGGACAACGCCCTGCAGTCCGGCAACTCCCAGGAATCCGTCAC |
| | | | CGAGCAGGACTCCAAGGACAGCACCTACTCCCTGTCCTCCACCCT |
| | | | GACCCTGTCCAAGGCCGACTACGAGAAGCACAAAGTGTACGCCT |
| | | | GCGAAGTGACCCACCAGGGCCTGTCCAGCCCCGTGACCAAGTCC |
| | | | TTCAACCGGGGCGAGTGC |
| 33 | 22 | Heavy Chain | GATATCGCCACCATGGCCTC TCCAGCTCAG CTGCTGTTTC |
| | | DNA (5′ and | TGCTGCTGCTGTGGCTGCCTGACGGCGTGCACGCACAAGTGCAGT |
| | | 3′ EcoRV | TGGTGCAGTCCGGAGCCGAAGTCAAGAAGCCCGGGTCGAGCGTG |
| | | restriction | AAAGTGTCCTGCAAGGCTTCTGGTGGAACCTTCTCAAGCGAAGG |
| | | sites (bold), | GATCAGCTGGGTCAGACAGGCGCCGGGCCAGGGTCTGGAGTGGA |
| | | Kozak | TGGGTTCCATTCTCCCGATCTTCGGAACCGCCAATTACGCCCAGA |
| | | sequence | AGTTCCAGGGTCGCGTGACCATCACCGCCGACGAAAGCACCTCG |
| | | (italics); | ACGGCCTATATGGAATTGTCGTCCCTGCGGTCGGAAGATACAGCG |
| | | signal | GTGTACTACTGTGCGCGGGAAGCCGGGTACTACCGCTACCGCTAC |
| | | sequence | TTCGATCTGTGGGGAAGGGGAACTCTCGTGACTGTGTCGAGCGCC |
| | | (underline)) | AGCACCAAGGGACCCAGCGTGTTCCCGCTGGCCCCTTGTTCACGA |
| | | | TCCACTTCCGAAAGCACCGCTGCCCTTGGCTGCCTTGTCAAGGAC |
| | | | TACTTCCCTGAGCCCGTCACTGTGTCGTGGAACAGCGGAGCTCTG |
| | | | ACCTCCGGCGTCCACACCTTCCCGGCTGTGCTCCAGTCCTCCGGC |
| | | | CTGTACTCACTGTCCTCGGTGGTCACCGTGCCCTCCTCCTCCCTCG |
| | | | GTACCAAGACTTATACCTGCAACGTGGACCACAAGCCCTCCAAC |
| | | | ACCAAAGTGGATAAGAGAGTGGAGAGCAAATACGGACCTCCCTG |
| | | | CCCTCCTTGCCCTGCGCCTGAGTTTCTGGGCGGACCATCCGTCTTT |
| | | | CTGTTCCCACCGAAGCCCAAGGACACCCTCATGATCTCCCGGACC |
| | | | CCCGAAGTGACCTGTGTGGTGGTGGACGTGTCACAGGAGGACCC |
| | | | TGAAGTGCAGTTTAATTGGTACGTCGACGGCGTGGAAGTGCATA |
| | | | ACGCAAAGACCAAGCCGCGGGAGGAACAGTTCAACTCAACCTAC |
| | | | CGCGTGGTGTCCGTGCTGACTGTGCTGCACCAGGACTGGCTGAAC |
| | | | GGAAAGGAGTATAAGTGCAAAGTCTCCAACAAGGGACTGCCGAG |
| | | | CAGCATCGAGAAAACCATTTCAAAAGCCAAGGGCCAGCCGAGAG |
| | | | AGCCCCAAGTGTACACTCTGCCGCCGAGCCAAGAGGAAATGACC |
| | | | AAGAACCAAGTGTCCCTCACTTGCCTGGTCAAGGGCTTCTACCCG |
| | | | TCGGACATCGCCGTGGAGTGGGAAAGCAACGGCCAGCCGGAAAA |
| | | | CAACTACAAGACTACCCCTCCCGTCCTCGACTCCGACGGGTCCTT |
| | | | CTTCCTCTACTCCCGGCTGACTGTGGATAAGTCACGGTGGCAGGA |
| | | | GGGAAACGTGTTCTCGTGCTCCGTGATGCACGAAGCCCTGCACAA |
| | | | CCACTACACGCAGAAGTCCCTGTCCTTGTCCCTGGGGAAGTAATG |
| | | | AGATATC |

1. A method of treating cancer in a human subject in need thereof comprising administering (i) a pharmaceutical composition comprising an IgG4 anti-CD39_229p antibody and (ii) a pharmaceutical composition comprising pembrolizumab or a pembrolizumab variant, wherein the IgG4 anti-CD39_229p antibody is administered at a dose of 20, 70, 200, 700, 1400, or 2000 mg.

2. The method of claim 1, wherein:
(a) the IgG4 anti-CD39_229p antibody is administered intravenously; or
(b) the IgG4 anti-CD39_229p antibody is administered once every 1, 2, 3, 4, 5 or 6 weeks; or
(c) the IgG4 anti-CD39_229p antibody is administered once every 2 weeks; or
(d) the pembrolizumab or pembrolizumab variant is administered at a dosage of 200 mg every 3 weeks; or
(e) the pembrolizumab or pembrolizumab variant is administered at a dosage of 400 mg every 6 weeks; or
(f) the pembrolizumab or pembrolizumab variant is administered intravenously.

3-7. (canceled)

8. A pharmaceutical composition comprising an IgG4 anti-CD39_229p antibody and a pharmaceutical composition comprising pembrolizumab or a pembrolizumab variant for use in treating cancer in a human subject in need thereof, wherein the IgG4 anti-CD39_229p antibody is administered at a dose of 20, 70, 200, 700, 1400, or 2000 mg.

9. The use of claim 8, wherein:
(a) the IgG4 anti-CD39_229p antibody is administered intravenously; or
(b) the IgG4 anti-CD39_229p antibody is administered once every 1, 2, 3, 4, 5 or 6 weeks;
or
(c) the IgG4 anti-CD39_229p antibody is administered once every two weeks; or
(d) the pembrolizumab or pembrolizumab variant is administered at a dosage of 200 mg every 3 weeks; or
(e) the pembrolizumab or pembrolizumab variant is administered at a dosage of 400 mg every 6 weeks; or
(f) the pembrolizumab or pembrolizumab variant is administered intravenously.

10-14. (canceled)

15. The method of claim 1, wherein:
(a) the cancer is newly diagnosed or non-metastatic; or
(b) the cancer is advanced; or
(c) the cancer is refractory; or
(d) the cancer is metastatic; or
(e) the cancer is a solid tumor; or
(f) the cancer is an advanced solid tumor; or
(g) the cancer is a relapsed solid tumor; or
(h) the cancer is a refractory solid tumor; or
(i) the cancer is a metastatic solid tumor; or
fj) the cancer is carcinoma, lymphoma, blastoma, sarcoma, or leukemia; or
(k) the cancer is pancreatic cancer; or
(l) the cancer is gastric cancer; or
(m) the cancer is prostate cancer; or
(n) the cancer is endometrial cancer; or
(o) the cancer is non-small cell lung cancer; or
(p) the cancer is colorectal cancer; or
(q) the cancer is ovarian cancer.

16-31. (canceled)

32. The method of claim 1, wherein:
(a) the cancer is selected from the group consisting of squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer (including squamous cell carcinoma of the head and neck); or
(b) the IgG4 anti-CD39_229p antibody and pembrolizumab or pembrolizumab variant are administered sequentially; or
(c) the pembrolizumab or pembrolizumab variant is administered before the IgG4 anti-CD39_229p antibody is administered; or
(d) the IgG4 anti-CD39_229p antibody is administered at a dose of 20 mg intravenously every 2 weeks; or
(e) the IgG4 anti-CD39_229p antibody is administered at a dose of 70 mg intravenously every 2 weeks; or
(f) the IgG4 anti-CD39_229p antibody is administered at a dose of 200 mg intravenously every 2 weeks; or
(g) the IgG4 anti-CD39_229p antibody is administered at a dose of 700 mg intravenously every 2 weeks; or
(1) the IgG4 anti-CD39_229p antibody is administered at a dose of 1400 mg intravenously every 2 weeks; or
(i) the IgG4 anti-CD39_229p antibody is administered at a dose of 2000 mg intravenously every 2 weeks.

33-40. (canceled)

41. The method of claim 1, wherein the IgG4 anti-CD39_229p antibody is administered at a dose of 20 mg intravenously every 2 weeks, and wherein pembrolizumab or the pembrolizumab variant is administered at a dose of 200 mg intravenously every 3 weeks.

42. The method of claim 1, wherein the IgG4 anti-CD39_229p antibody is administered at a dose of 70 mg intravenously every 2 weeks, and wherein pembrolizumab or the pembrolizumab variant is administered at a dose of 200 mg intravenously every 3 weeks.

43. The method of claim 1, wherein the IgG4 anti-CD39_229p antibody is administered at a dose of 200 mg intravenously every 2 weeks, and wherein pembrolizumab or the pembrolizumab variant is administered at a dose of 200 mg intravenously every 3 weeks.

44. The method of claim 1, wherein the IgG4 anti-CD39_229p antibody is administered at a dose of 700 mg intravenously every 2 weeks, and wherein pembrolizumab or the pembrolizumab variant is administered at a dose of 200 mg intravenously every 3 weeks.

45. The method of claim 1, wherein the IgG4 anti-CD39_229 p antibody is administered at a dose of 1400 mg intravenously every 2 weeks, and wherein pembrolizumab or the pembrolizumab variant is administered at a dose of 200 mg intravenously every 3 weeks.

46. The method of claim 1, wherein the IgG4 anti-CD39_229 p antibody is administered at a dose of 2000 mg intravenously every 2 weeks, and wherein pembrolizumab or the pembrolizumab variant is administered at a dose of 200 mg intravenously every 3 weeks.

47. The method of claim 1, wherein the IgG4 anti-CD39_229 p antibody is administered at a dose of 20 mg intravenously every 2 weeks, and wherein pembrolizumab or the pembrolizumab variant is administered at a dose of 400 mg intravenously every 6 weeks.

48. The method of claim 1, wherein the IgG4 anti-CD39_229 p antibody is administered at a dose of 70 mg intravenously every 2 weeks, and wherein pembrolizumab or the pembrolizumab variant is administered at a dose of 400 mg intravenously every 6 weeks.

49. The method of claim 1, wherein the IgG4 anti-CD39_229p antibody is administered at a dose of 200 mg intravenously every 2 weeks, and wherein pembrolizumab or the pembrolizumab variant is administered at a dose of 400 mg intravenously every 6 weeks.

50. The method of claim 1, wherein the IgG4 anti-CD39_229p antibody is administered at a dose of 700 mg intravenously every 2 weeks, and wherein pembrolizumab or the pembrolizumab variant is administered at a dose of 400 mg intravenously every 6 weeks.

51. The method of claim 1, wherein the IgG4 anti-CD39_229p antibody is administered at a dose of 1400 mg intravenously every 2 weeks, and wherein pembrolizumab or the pembrolizumab variant is administered at a dose of 400 mg intravenously every 6 weeks.

52. The method of claim 1, wherein the IgG4 anti-CD39_229p antibody is administered at a dose of 2000 mg intravenously every 2 weeks, and wherein pembrolizumab or the pembrolizumab variant is administered at a dose of 400 mg intravenously every 6 weeks.

53. A method of treating cancer in a human subject in need thereof comprising administering a pharmaceutical composition comprising an IgG4 anti-CD39_229p antibody, a pharmaceutical composition comprising gemcitabine, a pharmaceutical composition comprising paclitaxel, and a pharmaceutical composition comprising pembrolizumab or a pembrolizumab variant,
wherein the IgG4 anti-CD39_229p antibody is administered at a dose of 1400 mg intravenously every 2 weeks;
wherein the gemcitabine is administered at a dose of 1000 mg/m² intravenously on days 1, 8, and 15 of a 28-day cycle;
wherein the paclitaxel is administered at a dose of 125 mg/m² intravenously over 30 to 40 minutes prior to gemcitabine on days 1, 8, and 15 of a 28-day cycle; and
wherein the pembrolizumab or the pembrolizumab variant is administered at a dose of 200 mg intravenously every 3 weeks.

54. The method of claim 53, wherein the cancer is pancreatic cancer or wherein the paclitaxel is albumin-bound paclitaxel.

55. (canceled)
